(19) United States
(12) Patent Application Publication
(10) Pub. No.: US 2012/0004398 A1
(43) Pub. Date: Jan. 5, 2012

(54) Method for Uses of Protein Precursors as Prodrugs

(75) Inventors: Wei-Chiang SHEN, San Marino, CA (US); Yan WANG, Alhambra, CA (US)

(73) Assignee: UNIVERSITY OF SOUTHERN CALIFORNIA, Los Angeles, CA (US)

(21) Appl. No.: 13/174,520

(22) Filed: Jun. 30, 2011

Related U.S. Application Data
(60) Provisional application No. 61/361,248, filed on Jul. 2, 2010.

(51) Int. Cl. C07K 19/00; C12P 21/02

(52) U.S. Cl. 530/394; 435/69.4

(57) ABSTRACT

The present invention provides compositions useful as prodrugs and methods for making the same. The compositions include a fusion protein having a first delivery domain and a second protein precursor domain linked together via a linker sequence. The delivery domain is a protein capable of facilitating entry to a target cells via the endocytotic pathway, such as transferrin. The protein precursor is a prohormone or a profactor, such as proinsulin. Methods of this invention include the steps of selecting a protein suitable as the delivery domain, constructing a vector to encode the fusion protein, and expressing the fusion protein in a suitable expression host.

### STATEMENT REGARDING FEDERALLY SPONSORED RESEARCH OR DEVELOPMENT

This invention was made with government support under Contract No. GM 063647 awarded by NIH and HIGMS. The government has certain rights in the invention.

### FIELD OF THE INVENTION

The invention pertains to the field of drug delivery and protein engineering. More particularly, the invention pertains to methods and compositions for delivering protein-based therapeutics, such as prohormones and profactors, without the need for in vitro chemical or proteolytic processing to produce therapeutically effective drugs.

### BACKGROUND OF THE INVENTION

A number of biologically active peptides or proteins, including hormones, cytokines, neuropeptides and growth factors, are initially generated in the form of larger, inactive precursor peptides. These precursor peptides, or propeptides, including prohormones and profactors, generally require specific intracellular proteolytic processing to turn them into their active forms for biological functions {1, 2}. In terms of protein manufacturing, the precursor forms of the peptides are often first synthesized instead of the mature forms. This is because the mature forms of the peptides often have complex conformations, low expression yield, or are structurally unstable. In terms of protein drug delivery, the propeptides, but not the mature peptides, are linked to another protein moiety through chemical conjugation or recombinant fusion to achieve specific delivery goals and enhance overall protein stability [3]. Therefore, in order to exhibit biological activity, the propeptides need to be processed and activated, which is an important and challenging step in the production of recombinant therapeutic proteins.

Conventional methods of delivering the prodrugs generally involve chemical conjugation to link together the propeptide with a delivery protein. However, the major obstacle for chemically conjugating the two domains is that the composition and size of the final product can be heterogeneous, which is unacceptable for therapeutic use. Therefore, there still exists a need for a better approach to form fusion proteins that link together a delivery domain with a prodrug domain.

### SUMMARY OF THE INVENTION

The present invention is based on the unexpected discovery that in transferrin receptor-mediated endocytosis, the intracellular compartments of hepatocytes and epithelial cells has the ability to convert proinsulin to insulin. Inspired by this discovery, the inventors have conceived and reduced to practice a method of formulating a protein-based drug by conjugating a propeptide of a protein factor with a tranferrin domain. For example, an insulin-based protein drug may be formed by conjugating the propeptide of insulin (i.e. proinsulin) with transferrin. The proinsulin-transferrin conjugate or fusion protein will be converted to fully active insulin-transferrin when incubated with hepatocytes. In addition to acting as an activation element, the transferrin moiety can further increase the stability and sustained activity of the insulin or other therapeutic peptides compare to their non-conjugated counterparts.

It will be recognized by those skilled in the art that many propeptides of protein factors, such as proinsulin and proglucagon, cannot be used as drugs unless they have been converted to the active peptides by chemical or proteolytic treatment to cleave specific peptide bonds, such as the removal of c-peptide from proinsulin. Alternatively, insulin can be synthesized by two-peptide recombinant method which involves the CNBr treatment to produce A- and B-chain separately followed by the oxidoreductive reaction to form the interchain disulfide bonds [4]. Such modification processes after the production of the propeptides is both technically-challenging and cost-inefficient for therapeutic peptide production. The present invention enables the use of propeptides as drugs without additional chemical or enzymatic processing.

Accordingly, in one aspect, the present invention provides a fusion protein useful as a prodrug. Fusion proteins in accordance with embodiments of this aspect of the present invention will generally having a first delivery domain linked to a second protein precursor domain via a linker sequence. The delivery domain is a protein capable of facilitating entry to a target cell via the endocytotic pathway. The second protein precursor domain is preferably a prohormone or a profactor.

In another aspect, the present invention also provides a method for delivering a protein precursor domain to a subject in need of said precursor domain. Methods in accordance with embodiments of this aspect of the present invention will generally include the steps of forming a fusion protein having a delivery domain linked to the protein precursor domain; and administering said fusion protein to the patient.

In yet another aspect, the present invention also provides a method for forming a fusion protein useful as a prodrug. Methods in accordance with embodiments of this aspect of the invention will generally include the steps of selecting a protein useful as a delivery domain for a protein precursor; constructing a vector encoding said delivery domain linked to said protein precursor via a suitable linker sequence; and expression said fusion protein in a suitable expression host.

In still another aspect, the present invention also provides a method for extending a protein precursor domain's half-life in plasma. Methods in accordance with embodiments of this aspect of the invention will generally include the steps of conjugating the protein precursor domain to a transferrin domain prior to introducing the protein precursor domain into the plasma. Here the transferrin domain acts as a half-life extending element to extend the plasma half-life of the protein precursor in the plasma.

In still a further aspect, the present invention also provides a method for extending a therapeutic effect of a protein precursor in a subject. Methods in accordance with embodiments of this aspect of the invention will generally include the steps of conjugating the protein precursor to a transferrin domain so as to form a fusion protein having the protein precursor domain linked to the transferrin domain via a linker sequence. Here the transferrin domain acts as a therapeutic effective stabilizing element that extends the therapeutic effective time of the protein precursor.

Other aspects and advantages of the invention will be apparent from the following description and the appended claims.

### BRIEF DESCRIPTION OF THE DRAWINGS

FIG. 1 illustrates the preproinsulin-Tf fusion gene construct in pcDNA 3.1 (+) vector.

FIG. 2 shows the results of Western blot of PI-Tf fusion protein using anti-Tf and anti-(pro)insulin antibodies, demonstrating the expression of both Tf and proinsulin in the fusion protein. Lane 1 to 5 is anti-Tf blot, and lane 7 is anti-(pro)insulin blot. Lane 1: 5 ng apo-Tf. Lane 2: 20 ng apo-Tf. Lane 3: PI-Tf. Lane 4: 5 ng apo-Tf+PI-Tf. Lane 5: 20 ng apo-Tf+PI-Tf. Lane 6: Marker. Lane 7: PI-Tf.

FIG. 3 shows graphs of the production of glucose by hepatoma H4IIE cells. They demonstrate the activity of PI-Tf fusion protein in inhibiting hepatic glucose production is higher than that of insulin and proinsulin. Furthermore, FIG. 3B shows that the activity of PI-Tf can be abolished in the presence of a large excess of Tf, suggesting that the activity is mediated by UR binding. (A) Inhibition curve of proinsulin, insulin and PI-Tf fusion protein. (B) Increased hepatic glucose production was blocked by 1000-fold Tf co-incubation.

FIG. 4 shows graphs for the measurement of insulin in the solutions. They demonstrate the conversion of PI-Tf to insulin-Tf in the presence of hepatoma H4IIE cells.

FIG. 5 shows a bar graph illustrating the competition of insulin receptor binding. It demonstrates the improved insulin receptor binding affinity of PI-Tf fusion protein which is preincubated in H4IIE cells under 37° C.

FIG. 6 shows a bar graph of glucose uptake by adipocytes. It demonstrates the uptake of 2-deoxy-D-[2, 6-3H] glucose into cultured adipocytes is stimulated by H4IIE-pretreated PI-Tf fusion protein. (A) Dose-dependent curve of glucose uptake stimulation by insulin and proinsulin. (B) Comparison of glucose uptake by 100 nM insulin, proinsulin, proinsulin and Tf in equamolar ratio, and PI-Tf fusion protein. (C) H4IIE-pretreated PI-Tf fusion protein exhibited increased activity, compared with non-cell-treated fusion protein. Asterisk indicated p<0.01 evaluated from t-test.

FIG. 7 shows a comparison of in vivo pharmacokinetic profiles of an exemplary ProINS-Tf fusion protein and ProINS.

FIG. 8 shows a comparison of the in viva hypoglycemic efficacy of an exemplary ProINS-Tf fusion protein against PBS, INS, and ProINS.

### DETAILED DESCRIPTION

As used herein, the term “protein precursor” refer to inactive proteins or peptides that can be turned into an active form by posttranslational modification. Exemplary “protein precursor” may include proinsulin, proglucagon and proopiomelanocortin, but are not limited thereto.

As used herein, the term “prodrug” refers to a pharmacological substance that is administered in an inactive or significantly less active form, but becomes activated in viva through metabolic activities either intracellularly or extracellularly. Exemplary prodrugs may include prohormones and other profactors.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless

Unless otherwise indicated, all terms used herein have the meanings given below, and are generally consistent with same meaning that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel F M et al. (1993) Current Protocols in Molecular Biology, John Wiley & Sons, New York, N.Y., for definitions and terms of the art. It is to be understood that this invention is not limited to the particular methodology, protocols, and reagents described, as these may vary.

The term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. Accordingly, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

All publications cited herein are expressly incorporated herein by reference for the purpose of describing and disclosing compositions and methodologies that might be used in connection with the invention.

Proteins suitable as the delivery domain will depend on the target cells. Preferably, the protein is one that can facilitate entry to the target cell with the endocytotic pathway,

The linker sequence is preferably short and stable. In some embodiments, the linker is resistant to protyolytic cleavage so that the fusion protein remain intact in vivo. In other embodiments, the linker sequence is designed to be cleaved under suitable environments, such as under acidic or proteolytic conditions of the endocytic vesicle.

As a demonstration, the inventors have obtained a fusion protein of proinsulin-transferrin and have demonstrated that the fusion protein can be converted to insulin-transferrin in liver cell cultures. Unlike the inactive proinsulin, proinsulin-transferrin fusion protein, after incubated with liver cells, possesses higher activity in gluconeogenesis and equal activity in glucose transport when compared to active insulin. Thus, demonstrating that a fusion protein in accordance with embodiments of this invention are useful as prodrugs.

The present invention will now be further illustrated by referring to specific embodiments as shown in the following examples and the accompanying figures. It will be understood that the following examples are provided in order to demonstrate and further illustrate certain embodiments and aspects of the present invention and are not to be construed as limiting the scope thereof. Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the appended claims.

### EXAMPLES

### Example 1

### PI-Tf Recombinant Fusion Protein Expression and Characterization

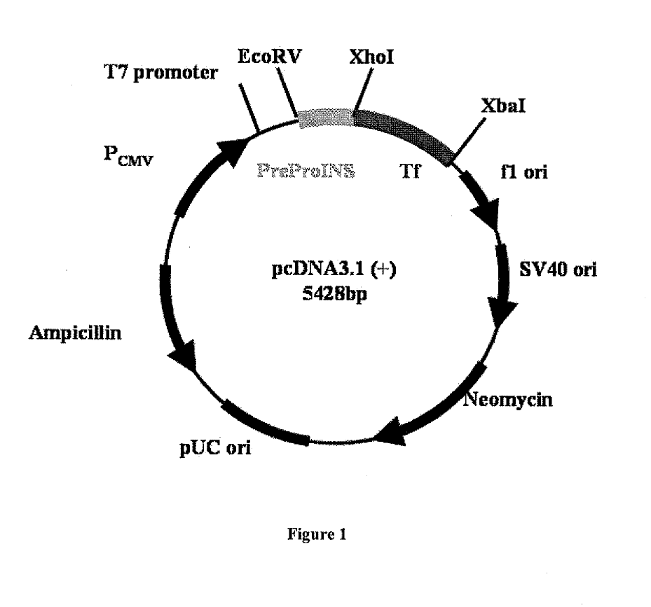

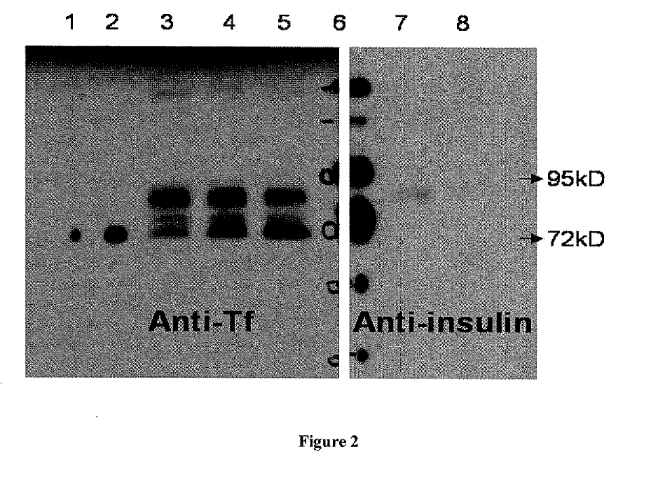

Preproinsulin sequence (NM_{—}000207) fused in frame with Tf sequence (NM 001063) was engineered into pcDNA3.1 (-9 expression vector (Invitrogen, Calif.) by molecular cloning methods (FIG. 1). Plasmids containing preproinsulin-Tf fusion gene were transiently transfected to HEK 293 cells through polyethylenimine-mediated DNA transfection. Conditioned serum-free media were collected and concentrated by labscale tangential flow filtration system (Millipore, Mass.), and then ultrafiltered by Centricon (Millipore, Mass.). PI-Tf fusion protein was characterized and quantified by Western blot using both anti-Tf (Sigma, Mo.) and anti-(pro)insulin antibodies (Abeam, MA). Anti-Tf and anti-(pro)insulin Western blots demonstrated the presence of a major band with molecular weight ˜89 kD, which indicated that PI-Tf fusion protein was successfully expressed and secreted into media. A leucine-glutamate dipeptide sequence was introduced between proinsulin and If due to the XhoI restriction enzyme cutting site. The Tf shown on Lane 3 of FIG. 2 came from the original serum-free cell culture medium, CD 293 (Invitrogen), instead of production from transfected HEK293 cells. The dipeptide linker remained stable during production process.

### Example 2

Enhanced Inhibition of Hepatic Glucose Production by PI-If Fusion Protein in H4IIE Hepatoma Cells

Rat hepatoma H4IIE cells were cultured in high-glucose DMEM containing 10% fetal bovine serum. Upon confluency, cells were treated with different drugs for 24 hrs at 37° C. Cells were washed twice with phosphate buffered saline. Glucose production media consisting of serum-, glucose- and phenol red-free DMEM supplemented with 2 mM sodium pyruvate and 40 mM LD-sodium lactate were added to cells for additional 3-hr incubation. The supernatant was harvested and applied to measure glucose concentrations using the Amplex Red Glucose/Glucose Oxidase kit (Invitrogen, Calif.) [5]. Cells were lysed in 1 M NaOH, and protein amount was quantified by BCA (Thermo Scientific, Ill.).

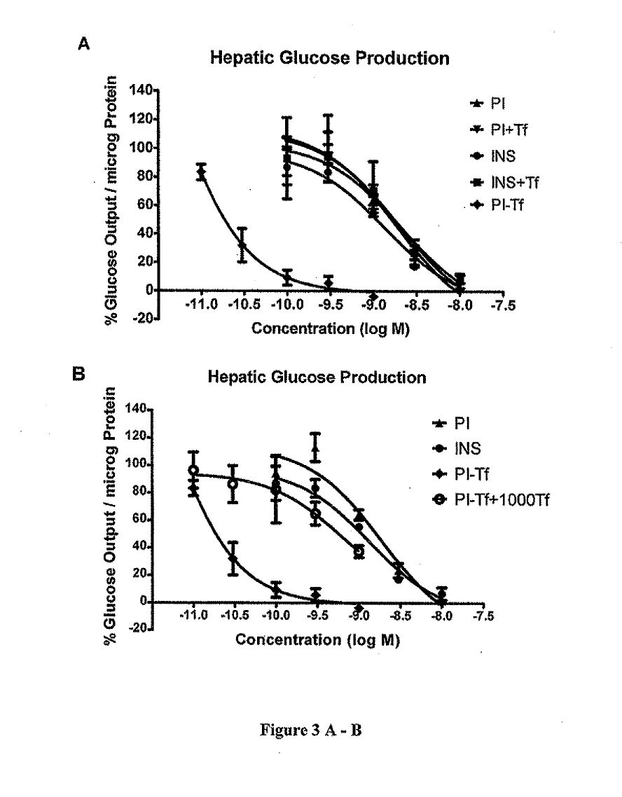

Proinsulin and insulin exhibited comparable inhibitory activities in glucose production with IC₅₀ values of 1441.3±641.6 pM and 1093.9±105.6 pM, respectively (FIG. 3A). Proinsulin bound to insulin receptor, but it had a considerably lower binding affinity than insulin [6]. However, the higher stability of proinsulin allowed itself a slower degradation rate, which may result in similar activity as insulin in the 24 hr-incubation assay. PI-Tf fusion protein, with an IC₅₀ value of 4.60±5.78 pM, exerted ˜300-fold stronger activity than proinsulin and insulin. However, equimolar mixture of proinsulin/insulin and Tf did not significantly increase the inhibitory activity as fusion protein did. It is consequently suggested that the enhanced inhibition was due to the fusion of the two moieties. Co-incubation of fusion protein with excess Tf (1000-fold) was able to block the increased inhibition, allowing the activity to reduce to that of insulin and proinsulin. However, no blocking effects were observed with excess albumin incubation (FIG. 3B). Therefore, introducing a Tf moiety to proinsulin as one single fusion protein can significantly enhance proinsulin's hepatic glucose inhibitory capacity.

### Example 3

### Conversion of PI-If to Insulin-If Fusion Protein by Hepatoma Cells

Rat hepatoma H4IIE cells (ATCC, VA) were treated with PI-Tf fusion protein in

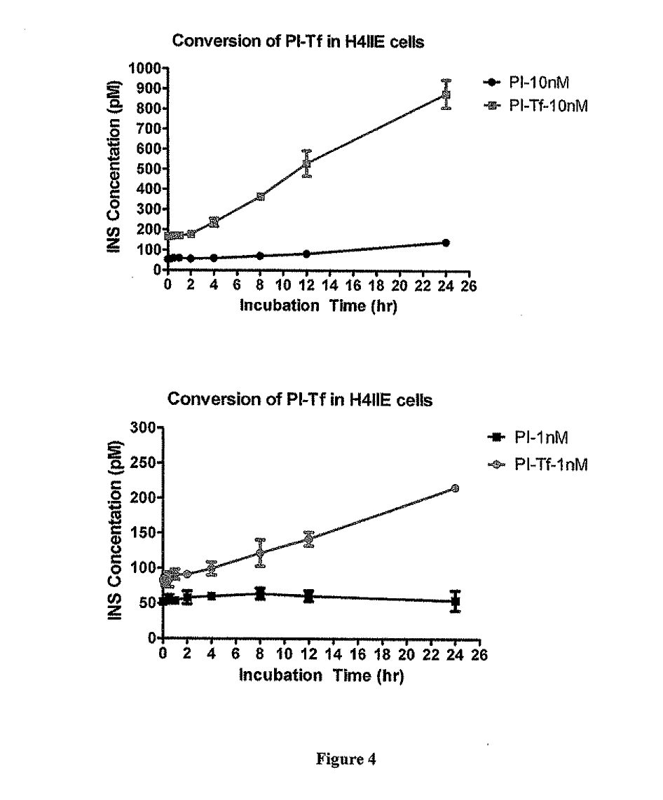

DMEM medium and incubated at 37° C. Media were collected at different time points, and subjected to insulin- and proinsulin-specific radioimmunoassays (Millipore, Mass.). Proinsulin and insulin concentrations were obtained based on standard curves from radioimmunoassays. After treatment in H4IIE cells for up to 24 hr, an insulin-containing species was continuously generated from PI-Tf fusion protein-treated samples, but not proinsulin-treated samples (FIG. 4). The generated insulin-containing species was suggested to be insulin-Tf instead of released insulin, since the two moieties were linked through stable peptide bonds. The conversion efficiency of PI-Tf to insulin-Tf was estimated 8.8% when dosed with 10 nM PI-Tf and 21.6% when dosed with 1 nM PI-Tf. These results demonstrated that the prohormone fusion protein PI-Tf can be converted to insulin-Tf in hepatoma cells, It also indicated that this hepatic conversion process was mediated by Tf, presumably occurring inside the intracellular recycling compartments during TfR-mediated endocytosis and recycling.

### Example 4

### Pretreatment of PI-Tf Fusion Protein in H4IIE Hepatoma Cells Leads to Increased Receptor Binding Affinity and Enhanced Stimulation of Glucose Transport

Insulin receptor binding assay was performed using H4IIE hepatoma cells [7]. [125I]-TyrA14 insulin (Perkin Elmer, Mass.) and various concentrations of unlabeled fusion proteins were treated to cells at 4° C. for 2 hr. Cells were washed twice with phosphate-buffered saline, and lysed with 0.1 N NaOH at RT. Radioactivity of total cell lysates was measured by gamma-counter. Protein amount were quantified by BCA assays.

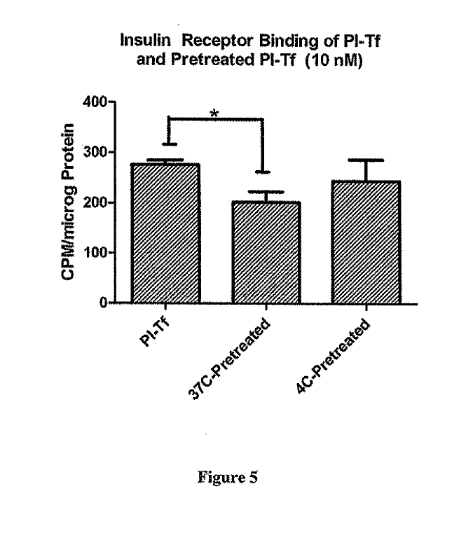

Compared with insulin, proinsulin exhibited a ˜100-fold lower binding affinity to insulin receptor on H4IIE hepatoma cells. In order to validate the hepatic conversion of PI-Tf to insulin-Tf, the binding affinity of H4IIE-pretreated PI-Tf fusion protein was measured. Briefly, cells were first treated with P1-Tf at either 37° C. or 4° C. for 1 hr, and subsequently equilibrated at 4° C. for 15 min prior to the addition of [1251]-TyrA14 insulin. Cells were then incubated for 2 hr at 4° C. to allow sufficient binding. Compared with non-treated fusion protein, 37° C.-pretreated PI-Tf fusion protein solutions exhibited a significantly increased binding capacity (FIG. 5). It is suggested that the increased binding might result from insulin-Tf generated during pretreatment in hepatoma cells. Besides, no significant changes were observed for the 4° C.-pretreated PI-Tf. These data indicated that the hepatic conversion to insulin-Tf was not processed by proteases on the cell membrane, whereas it required a cellular internalization to allow intracellular enzymatic processing of PI-Tf fusion protein. The internalization process was suggested to be facilitated through Tf-TfR-mediated endocytosis and recycling.

Insulin is known to promote glucose uptake in muscles and adipose tissues. To test whether PI-Tf and H4IIE-pretreated PI-Tf are active in glucose uptake stimulation, a glucose uptake assay was established using differentiated adipocytes as described previously [8]. Briefly, preadipocytes (murine 3T3-L1 fibroblasts) were induced to differentiate by a hormone cocktail consisting of bovine insulin, dexamethasone and 3-isobutyl-1-methylxanthine. After 10-14 days, cells reached full differentiation, Adipocytes were serum-starved prior to experiments. Cells were incubated with different drugs for 30 min in Krebs-Ringer phosphate (KRP) buffer supplemented with 0.1% bovine serum albumin. Glucose uptake was measured by the addition of 2-deoxy-D-[2, 6-3H] glucose (Perkin Elmer, Mass.). The reaction was stopped after 10 min by aspiration, and cells were washed four times with ice-cold KRP buffer. Cells were lysed with 0.1 M NaOH/0.1% SDS in KRP. Radioactivity was quantified by scintillation counting. Results were normalized for protein amount measured by BCA assays, For PI-Tf pretreatment in H4IIE cells, 10 nM of fusion protein solutions were dosed to H4IIE cells. After 24 hr incubation, the protein solutions were centrifuged, and the supernatants were collected to evaluate their activity of glucose uptake stimulation in adipocytes.

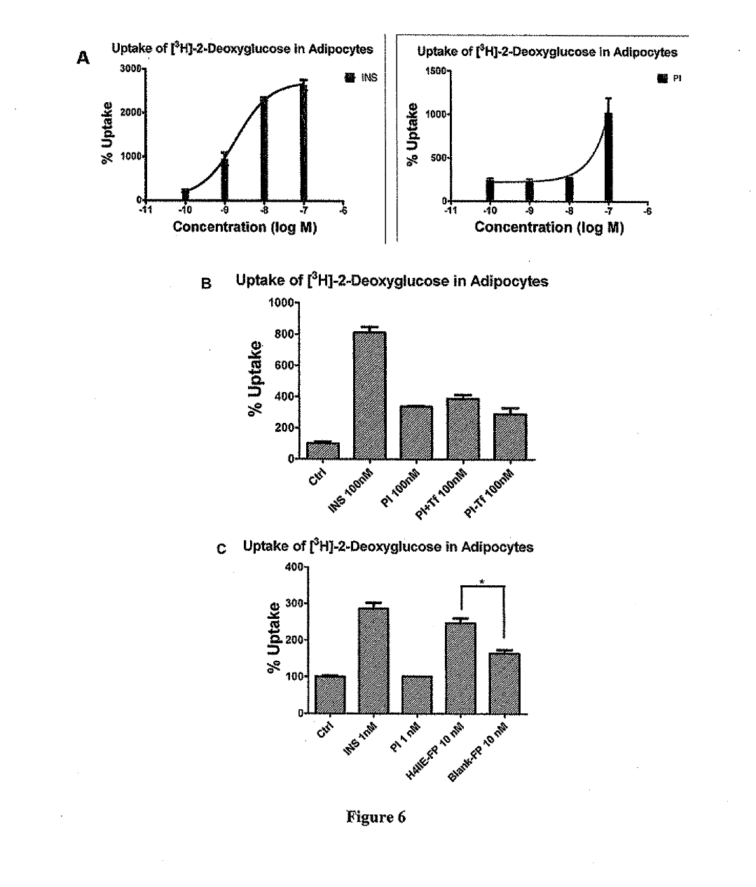

Insulin exhibited a strong stimulation in glucose uptake with EC50 values of 2 nM, whereas proinsulin was much less active (FIG. 6A). This is due to the much lower binding affinity of proinsulin to insulin receptor. Similar to proinsulin, PI-Tf fusion protein also exerted low stimulatory activity for the 30-min glucose uptake (FIG. 6B). However, when PI-Tf fusion protein pretreated in H4IIE cells was used to treat adipocytes, there showed a significantly increased glucose uptake, compared with PI-Tf pretreated in blank wells under the same experimental conditions (FIG. 6C). This result demonstrated that the insulin-Tf converted by hepatoma cells was biologically active in stimulating glucose uptake. Therefore, hepatic pretreatment can sufficiently convert and activate Pl-Tf fusion proteins. These data implied the application of PI-Tf fusion proteins as a prodrug for treatment of diabetes through either invasive or non-invasive delivery routes.

### Example 5

Prolonged in vivo Plasma Half-life of ProINS-Tf Fusion Protein

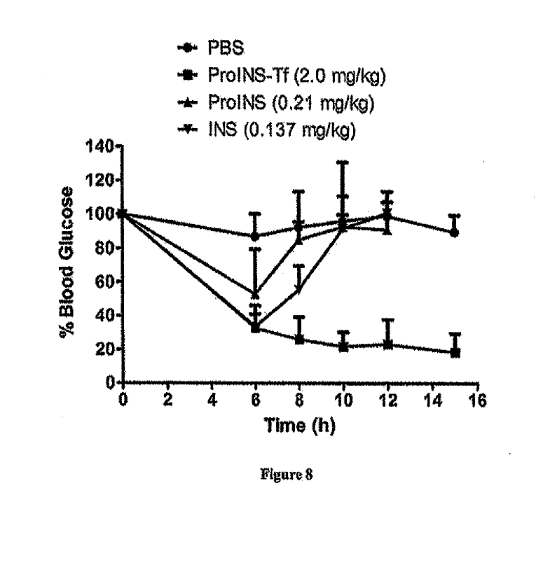

A single dose of 0.5 mg/kg ProINS-Tf or 0.053 mg/kg ProINS was injected intravenously to CF-I mice. Plasma concentrations of ProINS-Tf and ProINS were measured by ProINS-specific RIA (Millipore, Mass.). Data were obtained from 4 mice and shown in FIG. 7.

In vivo pharmacokinetics. Male CF-1 mice (6-7 weeks old) were fasted for 6 h prior to drug administration. A single dose of ProINS-Tf or ProINS was injected intravenously. Blood was sampled at different time points through saphenous veins. Whole blood was mixed with heparin and centrifuged to collect plasma. Plasma concentrations of ProINS-Tf and ProINS were determined by ProINS-specific RIA using ProINS-Tf and ProINS as standard curve, respectively.

### Example 6

Sustained and Enhanced in vivo Hypoglycemic Efficacy of ProINS-Tf Fusion Protein

STZ-induced diabetic mice were given a single subcutaneous injection of PBS, INS, ProINS, or ProINS-Tf fusion proteins with the same molar dose. Mice were fasted during experiments. Blood glucose levels were measured using OneTouch glucose meter. All the time points indicate hours post-injection. Data are expressed as the percentage of blood glucose compared to 0 h (initial blood glucose levels prior to injection). FIG. 8 and the following table summarizes the result of the experiment. Data represent averages±standard deviation (N=3-5).

| Injection Groups | 6 h | 8 h | 10 h | 12 h |
|---|---|---|---|---|
| PBS | 87.1 ± 13.2 | 92.5 ± 3.25 | 96.2 ± 3.67 | 99.2 ± 8.22 |
| INS (3 U/kg, 0.137 | 33.4 ± 7.58 | 55.5 ± 14.1 | 91.7 ± 39.3 | 109.9 ± 12.8 |
| mg/kg) | | | | |
| ProINS (0.21 mg/kg) | 52.7 ± 26.6 | 85.1 ± 28.5 | 92.9 ± 18.1 | 91.2 ± 16.4 |
| ProINS-Tf (2 mg/kg) | 32.6 ± 13.4 | 26.0 ± 13.3 | 21.8 ± 8.44 | 23.1 ± 14.7 |

In vivo hypoglycemic efficacy. Male C57BL/6J mice (6-7 weeks old) were given a single intraperitoneal injection of 175 mg/kg streptozotocin. Six days post-injection, mice became diabetic with fasting blood glucose levels ˜500 mg/dL. Diabetic mice were fasted for 2 h prior to a single subcutaneous injection of proteins. Blood was sampled through tail veins at various time points. Blood glucose levels were measured using OneTouch glucose meter.

### REFERENCES

The following cited references are each incorporated herein by reference.
[1] Rholam M and Fahy C. Processing of Peptide and Hormone Precursors at the Dibasic Cleavage Sites. Cell. Mol. Life Sci. 2009, 66, 2075-2091.
Smeekens S. P. Processing of Protein Precursors by a Novel Family of Subtilisin-Related Mammalian endoproteases. Nat. Biotech 1993, 11, 182-186.
Wurm F. M. Production of Recombinant Protein Therapeutics in Cultivated Mammalian Cells. Nat. Biotech., 2004, 22, 1393-1398.
Wong, D. W. S. Microbial Production of Recombinant Human Insulin. The ABCs of Gene Cloning (2^{nd} Ed), 2007, Springer US, pp. 159-162.
Raemy-Schenk A-M., Trouble S., Gaillard P. et al. A Cellular Assay for Measuring the Modulation of Glucose Production in H4IIE Cells. Assay Drug Dev. Tech. 2006, 4(5), 525-533.
Levy, J. R., Ullrich, A., Olefsky, J. M. Endocytotic Uptake, Processing, and Retroendocytosis of Human Biosynthetic Proinsulin by Rat Fibroblasts Transfected with the Human Insulin Receptor Gene.J. Clin. Invest. 1988, 81, 1370-1377.
Johansson G. S. and Arnqvist H. J. Insulin and IGF-1 Action on Insulin Receptors, IGF-1 Receptors, and Hybrid Insulin/IGF-1 Receptors in Vascular Smooth Muscle Cells. Am. J. Physiol. Endocrinol. Metab. 2006, 291, E1124-E1130.
Harmon A. W., Paul D. S., Patel Y. M. MEK Inhibitors Impair Insulin-Stimulataed Glucose Uptake in 3T3-L1 Adipocytes. Am. J Physiol. Endocrinol. Metab. 2004, 287, E758-E766.

What is claimed is:

1. A fusion protein useful as a prodrug, comprising:
a first delivery domain linked by a linker sequence to a second protein precursor domain comprising a protein precursor useful as a therapeutic agent,
wherein said first delivery domain is a protein capable of facilitating entry to a target cell via the endocytotic pathway, and said second protein precursor domain is a prohormone or a profactor.

2. The fusion protein of claim 1, wherein said first delivery domain is transferrin.

3. The fusion protein of claim 2, wherein said second protein precursor domain is proinsulin.

4. The fusion protein of claim 3, wherein said target cell is liver cell.

5. A method for delivering a protein precursor prodrug, comprising:
forming a fusion protein comprising a first delivery domain linked to a second protein precursor domain; and
introducing said fusion protein to a patient in need of the prodrug.

6. The method of claim 5, wherein said first delivery domain is a transferrin.

7. The method of claim 5, wherein said prodrug is proinsulin.

8. The method of claim 5, wherein said linker sequence is a leucine-glutamate dipeptide.

9. A method for forming a fusion protein useful as a prodrug, comprising:
selecting a protein useful as a delivery domain for a protein precursor;
constructing a vector encoding said delivery domain linked to said protein precursor via a suitable linker sequence; and
expressing said fusion protein in a suitable expression host.

10. The method of claim 9, wherein said delivery domain is transferrin.

11. The method of claim 9, wherein said protein precursor is proinsulin.

12. The method of claim 9, wherein said linker sequence is leucine-glutamate dipeptide.

13. A method for extending plasma half-life of a protein precursor domain, comprising:
conjugating said protein precursor domain to a transferrin domain prior to introducing said protein precursor into said plasma,
whereby said transferrin domain acts as a half-life extending element to extend the plasma half-life of the protein precursor domain.

14. The method of claim 13, wherein said protein precursor is a prohormone or a profactor.

15. The method of claim 13, wherein said protein precursor is proinsulin.

16. A method for extending a therapeutic effect of a protein precursor in a subject, comprising:
conjugating said protein precursor to a transferrin domain to form a fusion protein,
whereby said transferrin domain acts as a stabilizing element to extend the therapeutic effects of the protein precursor in the subject.

17. The method of claim 16, wherein said protein precursor is a prohormone or a profactor.

18. The method of claim 16, wherein said protein precursor is proinsulin.
